(19) United States
(12) Patent Application Publication
(10) Pub. No.: US 2013/0052697 A1
(43) Pub. Date: Feb. 28, 2013

(54) METHOD FOR HYDROLYTIC PRE-TREATMENT OF LIGNOCELLULOSIC AND PERENNIAL HERBACEOUS BIOMASS AND FOR PRODUCTION OF SACCHARIDE AND BIOETHANOL FROM THE PRE-TREATED BIOMASS

(71) Applicants: Hyeun Jong Bae, Gwangju (KR); Han Ki Shin, Seongnam-si (KR); Seung Gon Wi, Gwangju (KR); Hyung Gun Kim, Suncheon-si (KR)

(72) Inventors: Hyeun Jong Bae, Gwangju (KR); Han Ki Shin, Seongnam-si (KR); Seung Gon Wi, Gwangju (KR); Hyung Gun Kim, Suncheon-si (KR)

(73) Assignee: INDUSTRY FOUNDATION OF CHONNAM NATIONAL UNIVERSITY, Gwangju (KR)

(21) Appl. No.: 13/695,041

(22) PCT Filed: Apr. 29, 2011
(86) PCT No.: PCT/KR11/03221

§ 371 Date: Oct. 28, 2012

(30) Foreign Application Priority Data
Apr. 30, 2010 (KR) 10-2010-0040826

(51) Int. Cl. C12P 19/14; C08B 15/00

(52) U.S. Cl. 435/99; 127/37

(57) ABSTRACT

Disclosed is a method for the hydrolytic pre-treatment of lignocellulosic and perennial herbaceous biomass. By the method, a material suitable for use in the production of saccharides and biofuels can be prepared from lignocellulosic biomass such as pine wood and oak tree wood and perennial herbaceous biomass such as flame grasses and reeds. It is characterized by wet-triturating, microwaving and popping processes. Also, a method is provided for the production of saccharides and bioethanol from the pre-treated biomass.

### CROSS REFERENCE TO RELATED APPLICATIONS AND CLAIM OF PRIORITY

This patent application claims benefit under 35 U.S.C. 119(e), 120, 121, or 365(c), and is a National Stage entry from International Application No. PCT/KR2011/003221, filed on Apr. 29, 2011, which claims priority to Korean Patent Application number 10-2010-0040826, filed on Apr. 30, 2010, entire contents of which are incorporated herein by reference.

### BACKGROUND

1. Technical Field

The present invention relates to a method for the hydrolytic pre-treatment of lignocellulosic and perennial herbaceous biomass. More particularly, the present invention relates to a method for preparing a material suitable for use in the production of saccharides and biofuels from lignocellulosic wood biomass such as pine wood and oak wood and perennial herbaceous biomass such as flame grasses and reeds, characterized by wet-milling, microwaving and popping processes. Also, the present invention is concerned with a method for the production of saccharides and bioethanol from the pre-treated biomass.

2. Background Art

Processes for producing biofuels are grossly divided into material acquisition, pre-treatment, saccharification, fermentation and purification. Pre-treatment is a unit process which is economically and technologically very important in the overall procedure of ethanol production. The pre-treatment process is particularly indispensible for the saccharification of lignocellulosic biomass. The primary purpose of pre-treatment is to reduce the size of materials and to increase reactivity for saccharification, thus improving the efficiency of subsequent processes including the hydrolysis of polysaccharides and the fermentation of ethanol. Effective pre-treatment disturbs the crystallinity of fibrins to enlarge the surface area thereof, with a concomitant increase in reactivity with enzymes. The pre-treatment process is also responsible for the conversion of the xylan structure of hemicelluloses into the pentose xylose. However, the pre-treatment process must be conducted to suppress the production of by-products to a maximal extent some of which act to interfere with subsequent saccharification and fermentation processes.

To date, a number of physical and chemical methods have been proposed for the pre-treatment of lignocellulosic biomass, including steam explosion, alkaline treatment, treatment with sulfur dioxide, treatment with hydrogen peroxide, treatment with supercritical ammonia, an ammonia freeze explosion process, an ammonia recycled percolation process, and a thermochemical process.

Nonetheless, the physical pretreatment processes are economically unbeneficial because they proceed slowly, consume lots of energy and do not ensure efficient saccharification. Featuring the use of strong acid or alkali, the chemical pretreatment processes cost a great deal and are unsuitable for mass-scale processes. In addition, the chemicals used in the chemical pretreatment processes are so highly toxic as to corrode the facility. Also, the waste generated as a result of the reaction between the chemicals and lignin produce environmental pollution.

Particularly, steam explosion, one of the most widely used techniques, suffers from the disadvantage of degrading saccharides, allowing hemicelluloses to be recovered at a rate less than 65%. Steam explosion can promise only a low saccharification yield for both non-lignocellulosic and lignocellulosic biomass, and thus is used in combination with treatment with diluted acid or a low concentration of alkali to improve the yield.

As a result of the use of steam explosion and treatment with diluted acid in combination, lots of saccharification by-products are generated, including furan and furfural from pentoses and hexoses, acetic acid from the acetyl group of hemicelluloses, and various phenolic compounds from lignins. Typically, these materials act as inhibitors against fermentation. On the other hand, when steam explosion is combined with a chemical pre- or post-treatment, the acid or alkali used produces the secondary environmental pollution of waste acid or alkali in addition to corroding the facility. Thus, not only is it difficult to recycle the medium used in the process, but excessive investment is required to protect the facility from the acid or alkali.

The chemical composition of wood is highly variable across plant species, e.g., needle-leaf trees or broadleaf trees, etc. and age of the wood. Typically, lignocellulosic biomass is composed of cellulose (40˜50%), hemicelluloses (25˜35%) and lignin (15˜20%). Cellulose is a polysaccharide consisting of a number of β(1→4) linked D-glucose units that are also bonded to each other through inter- and intramolecular hydrogen bond or Van der Waals forces. Hemicellulose is a branching macromolecule constructed of pentose and cellulose units which are linked via β-1,4 linkage. In cell walls, hemicellulose serves as an adhesive between cellulose and lignin. Lignin is an insoluble, refractory polymer in which aromatic compounds such as phenylpropanoid units are connected to each other in a haphazard manner. In fact, the technical and economical difficulty in the preparation of biofuels from lignocellulosic and perennial herbaceous biomass is attributed to the higher content of lignin compared to starchy (cereals) and sugar-based biomass. This chemical trait increases the production cost of biofuels from lignocellulosic and perennial herbaceous plants over starchy or sugar-based plants.

In Korea, approximately 2.2 million tons of lignocellulosic waste is produced every year, which includes waste wood from construction sites (concrete forms, scaffolds, etc.) living environments and factory sites (pallets, etc) in amounts of 50%, 40% and 10%, respectively. This waste wood is now recycled at a rate of 33.3%, with a recycling rate of as low as 2.5% for the waste wood from living environments. The reason why lignocellulosic biomass is not utilized as a material for biofuels is attributed to the higher production cost of bioenergy than that of fossil fuels, particularly, to the high cost of pre-treatment and the high price of commercial enzymes used for saccharification. Scores of research and development has been conducted into the technology and processes for industrializing the production of biofuels from biomass, but success cases have not yet been reported.

The present inventors developed effective pre-treatment methods which can economize in the consumption of energy without causing environmental problems as disclosed in Korean Patent Application Nos. 10-2007-0102493 and 10-2010-0008497. Korean Patent Application No. 10-2007-0102493 pertains to a method for the production of saccharides and bio-ethanol from lignocellulosic biomass, including 1) pre-treating lignocellulosic biomass by popping; and 2) saccharifying or saccharifying and fermenting the pre-treated biomass. Korean Patent Application No. 10-2010-0008497 discloses a method for the production of saccharides or bio-ethanol from lignocellulosic biomass, including 1) pre-treating lignocellulosic biomass by wet triturating and popping; and 2) saccharifying or simultaneously saccharifying and fermenting the pre-treated biomass.

The popping process disclosed in the previous patent applications of the present inventors requires neither a steam generator indispensable for steam explosion, nor the provision of chemicals, but utilizes a relatively simple popping machine based on a direct fired burner which is operated without being accompanied by secondary environmental pollution and corrosion. Also, the popping process does not allow the production of fermentation inhibitors from pentoses, hexoses and lignins at all, and is relatively simple. Thus, its technology is highly advanced over that of steam explosion.

The methods of the patent applications are very effective in saccharifying annual herbaceous biomass such as corn stalks, rice straw, etc., but a decrease in the saccharification rate for lignocellulosic biomass such as perennial herbaceous plants or lumber.

Therefore, there is the need for a method for the effective pre-treatment of lignocellulosic biomass that can cut down on the consumption of energy and that produces neither materials inhibitory of saccharification and fermentation nor environmental problems.

### SUMMARY

Leading to the present invention, intensive and thorough research, conducted by the present inventors aiming to solve the problems encountered in the prior art, resulted in the fmding that hydrolytic pre-treatment including immersing, microwaving and popping increases the saccharification yield of lignocellulosic and perennial herbaceous biomass.

It is therefore an aspect of the present invention to provide a method for the enzymatic pre-treatment of lignocellulosic and perennial herbaceous biomass and a method for the production of bioethanol from the pre-treated biomass which can effectively improve the saccharification yield of lignocellulosic and perennial herbaceous biomass.

It is another aspect of the present invention to provide a method for the enzymatic pre-treatment of biomass and a method for the production of a saccharide and bioethanol from the pre-treated biomass which do not produce environmental pollution due to excluding the use of chemicals, and can cut down on the energy consumed during pre-treatment, compared to conventional methods.

It is a further aspect of the present invention to provide a method for the enzymatic pre-treatment of biomass and a method for the production of a saccharide and bioethanol from the pre-treated biomass which are simple and economically beneficial for the production of bioethanol so that they can be industrialized.

It is still a further aspect of the present invention to provide a method for the enzymatic pre-treatment of biomass and a method for the production of a saccharide and bioethanol from the pre-treated biomass which allow the lignocellulosic biomass such as pine wood and oak tree wood and perennial herbaceous biomass, such as flame grasses and reeds, which are very difficult to degrade, to be used as materials to produce saccharides and bioethanol.

The aspects of the present invention are not limited to the above-mentioned aspects, and other aspects which are not described will be clearly understood to those skilled in the art from the following description.

In accordance with an aspect thereof, the present invention provides a method for the hydrolytic pre-treatment of lignocellulosic and perennial herbaceous biomass, including preparing lignocellulosic biomass or perennial herbaceous biomass in the form of chips, said lignocellulosic biomass including pine wood and oak wood, said perennial herbaceous biomass including reeds and flame grasses; immersing the biomass in water; microwaving the hydrated biomass for 10˜40 min at 500 W˜800 W; dewatering the microwaved biomass; and popping the dewatered biomass under a pressure of 5˜30 kgf/cm² in a popping machine

In accordance with another aspect thereof, the present invention provides a method for the hydrolytic pre-treatment of lignocellulosic and perennial herbaceous biomass, including preparing lignocellulosic biomass or perennial herbaceous biomass in the form of chips, said lignocellulosic biomass including pine woods and oak wood, said perennial herbaceous biomass including reeds and flame grasses; triturating the biomass in a wetting manner; microwaving the wet triturated biomass for 10˜40 min at 500 W˜800 W; dewatering the microwaved biomass; and popping the dewatered biomass under a pressure of 5˜30 kgf/cm² in a popping machine.

In an embodiment, the immersing step includes immersing the biomass in water for 3 hours or longer and stirring 100 wt parts of the biomass in 300˜700 wt parts of water.

In an embodiment, the triturating step includes swelling the biomass for 12 hours in water and triturating the swollen biomass to defibration.

In a an embodiment, the popping machine includes a popping tank for accommodating the wet-triturated biomass, designed to maintain high temperatures and high pressures therein when heated; a direct fired burner for applying heat directly to the popping tank; a storage tank having a space for storing the popped biomass, into which a part of the popping tank is detachably attached; a motor for rotating the popping tank directly heated by the direct fired burner, whereby temperature and steam distribution can be maintained constant across the popping tank; and a controller for controlling the popping tank in terms of either or both pressure and temperature.

In an embodiment, the popping tank is equipped with a temperature sensor and/or a pressure sensor therein.

In accordance with a further aspect thereof, the present invention provides a method for production of a saccharide from lignocellulosic and perennial herbaceous biomass, including saccharifying the lignocellulosic and perennial herbaceous biomass pre-treated by the method of one of claims 1 to 4.

In an embodiment, the saccharifying step is carried out by treating 100 weight parts of the biomass with 1˜20 weight parts of a sugar saccharifying enzyme.

In an embodiment, the saccharifying enzyme is selected from the group consisting of cellulase, xylanase, β-glucosidase and a combination thereof.

In accordance with still a further aspect thereof, the present invention provides a method for producing bioethanol from lignocellulosic and perennial herbaceous biomass, including: pre-treating the lignocellulosic and perennial herbaceous biomass using the method of one of claims 1 to 4; saccharifying the pre-treated biomass to give a saccharide; and fermenting the saccharide.

In an embodiment, the saccharifying step and the fermenting step are conducted simultaneously.

In an embodiment, the saccharifying step and the fermenting step are conducted simultaneously after the pre-treated biomass is treated with a recombinant strain selected from the group consisting of Klebsiella oxytoca P2, Brettanomyces curstersii, Saccharomyces uvzrun, and Candida brassicae.

One or more embodiments of the present invention enjoys the following advantages, but not limited thereto.

First, the method of the enzymatic pre-treatment of biomass according to the present invention can effectively improve the saccharification yield of lignocellulosic and perennial herbaceous biomass.

Also, the method for the enzymatic pre-treatment of biomass and the methods for the production of a saccharide and bioethanol from the pre-treated biomass in accordance with the present invention do not produce environmental pollution because they exclude the use of chemicals, and can cut down on energy consumed by pre-treatment, compared to conventional methods.

In addition, the method for the enzymatic pre-treatment of biomass and the methods for the production of a saccharide and bioethanol from the pre-treated biomass are simple and economically beneficial for the production of bioethanol so that they can be industrialized.

Moreover, the method for the enzymatic pre-treatment of the biomass and the methods for the production of a saccharide and bioethanol from the pre-treated biomass allow the lignocellulosic and perennial herbaceous biomass which is very difficult to degrade, such as the wood of needle-leaf trees (pine woods), the wood of broadleaf trees (oak wood), flame grasses and reeds, to be used as materials for the production of saccharides and bioethanol.

### BRIEF DESCRIPTION OF DRAWINGS

FIG. 1 is a schematic diagram showing the biological conversion of biomass into ethanol in accordance with the present invention.

FIG. 2 is a schematic view showing a popping machine in which a popping step is carried out in accordance with the present invention.

FIG. 3 is a FT-IR spectrum showing the cell wall components of the biomass pre-treated using the hydrolytic pre-treatment of the present invention.

FIG. 4 is a bar graph showing the enzymatic hydrolysis yield of each of the types of hydrolytically pre-treated biomass when the method for the production of a saccharide in accordance with the present invention is applied thereto.

FIG. 5 is an HPLC spectrum showing monosaccharides produced when the hydrolytically pre-treated biomass is enzymatically hydrolyzed in accordance with the present invention.

### DETAIED DESCRIPTION

The technical terms used in the present invention are adopted from general words currently widely used in the art, but may be used arbitrarily by the inventors in some cases. In this regard, the meanings of the terms must be understood in consideration of the context of the invention, but not in view of terms themselves.

Reference now should be made to the drawings, in which the same reference numerals are used throughout the different drawings to designate the same or similar components. A better understanding of the present invention may be obtained through the following embodiments and examples which are set forth to illustrate, but are not to be construed as limiting the present invention.

The present invention is technically characterized by a method for the hydrolytic pre-treatment of lignocellulosic biomass, particularly, lignocellulosic and herbaceous biomass whereby sugar compounds and/or bioethanol can be effectively produced. As seen in FIG. 1, the present invention is directed to a complex pre-treatment method in which wet triturating, microwaving and popping processes are sequentially conducted to remarkably increase the hydrolysis efficiency of lignocellulosic biomass. Instead of wet trituration, immersion in water, although not shown in FIG. 1, may be carried out

Therefore, the method for the hydrolytic pre-treatment of lignocellulosic and herbaceous biomass in accordance with an embodiment of the present invention includes preparing the lignocellulosic and herbaceous biomass in the form of chips, immersing the biomass in water or wet triturating the biomass, microwaving the biomass, dewatering the microwaved biomass, and popping the dewatered biomass.

The immersing step may be carried out by immersing the biomass in water for 3 hours or longer and for example, for 6 hours or longer and stirring 100 wt parts of the immersed biomass in 300 to 700 wt parts of water. The water is, for example, distilled water. The stirring may be performed using a stirrer, so that the biomass will absorb sufficient water. Thus, when the biomass is immersed for 12 hours or longer, with the weight ratio of biomass : water set at 1:3˜7, stirring may be omitted.

The wet triturating step includes swelling the biomass in water for 12 hours and grinding the swollen biomass to defibration. Lignocellulosic and perennial herbaceous biomass sufficiently swells in water and when finely mashed to defibration using a refiner or milling machine. The swelling is, for example, conducted by immersing the biomass in sufficient water for a day.

As for the microwaving step, it is conducted, for example, at 600 W to 800 W for 10 to 40 min in a microwave oven. When the biomass is microwaved at less than 600 W, it does not undergo a physical and structural change sufficient to facilitate the hydrolysis thereof On the other hand, when the biomass is treated at over 800 W, the microwaving power exceeding 800 W has no significant influence on the hydrolysis of biomass.

After the microwaving step, the dewatering step is conducted to separate water from the biomass to such an extent that the water content of the biomass is maintained at from 60 to 80%.

Using a popping machine, the popping step is carried out at a temperature of from 150 to 250° C. and/or under a pressure of from 5 to 30 kg/cm² and, for example, at a temperature of from 170 to 250° C. and/or under a pressure of from 15 to 25 kg/cm².

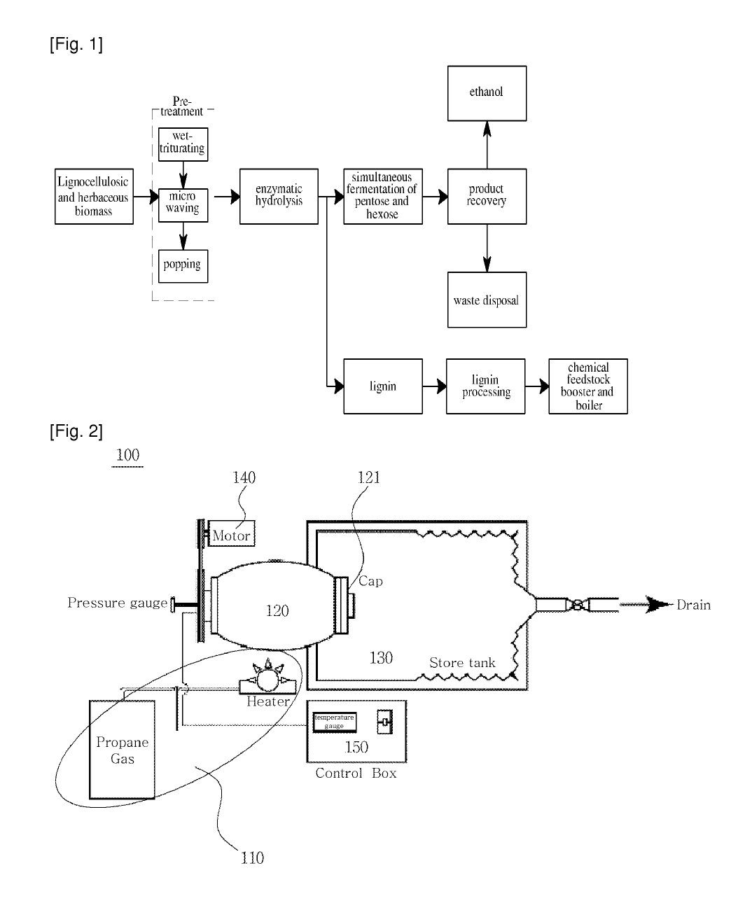

Referring to FIG. 2, the structure of the popping machine useful in the present invention is presented. As seen in FIG. 2, the popping machine 100 developed for conducting the popping step of the present invention includes a direct fired burner 110, a popping tank 120, a storage tank 130, a motor and a controller 150.

The direct fired burner 110 corresponds to a steam generator for steam explosion. The steam explosion has an indirect heating structure in which a steam generator is communicated with an explosion tank via a steam jacket to maintain high temperature and high pressure in the explosion tank. In the popping machine 100 of the present invention, the direct fired burner 110, like a gas burner including a gas tank and a heater, applies heat directly to the popping tank 120 to maintain a high temperature and a high pressure in the popping tank 120 and thus is much more advantageous than a steam generator used for steam explosion in terms of heat utility and safety.

The popping tank 120 is a container for accommodating the dewatered biomass and is, for example, made of a material which can endure high temperatures and pressures and which allows the direct application of heat thereto. The popping tank 120 is fixed at its one end to a frame in such a way that it is rotated by a motor 140, and has at the other end a cap-sealed opening 121 through which wet-triturated biomass is introduced into and drained out of the popping tank 120. For example, the opening is equipped with a hatch which functions to belch out the steam contained within the material after popping.

Inside the popping tank 120, a temperature sensor (not shown) is provided for detecting the temperature of the popping tank 120 and for transmitting the detected temperature data to a controller 150. A pressure gauge is installed outside the popping tank 120. Alternatively, a pressure sensor, instead of the pressure gauge, may be provided inside the popping tank 120.

The storage tank 130 is a component having a volume into which the material is discharged from the popping tank. For example, as shown, the popping tank 120 is detachably attached into the storage tank 130 in such a way that a part of the popping tank 120 is introduced into the storage tank 130 so as to reduce the popping sound. An outlet may be installed so as to discharge the popped biomass from the popping tank 120 into the storage tank 130.

A motor 140 is operated to rotate the popping tank 120 so that when the popping tank 120 is heated by the direct fired burner 110, a constant temperature and steam distribution is maintained throughout the popping tank 120.

The controller 150 is in the form of a control box including a keypad, a display window, sensors such as temperature gauge and pressure gauge and other elements to control, and functions to control the motor 140 and to open and close a valve installed between the gas tank and the heater of the direct fired burner 110 at set pressures and/or temperatures.

In the following Example Section, pine woods, oak wood, flame grasses, and reeds were used as lignocellulosic and perennial herbaceous biomass. However, so long as it is lignocellulosic or perennial herbaceous biomass, any material may be used in the present invention. Hence, the scope of the present invention is not limited by the lignocellulosic and perennial herbaceous biomass.

The saccharification of the pre-treated biomass may be acid saccharification, but is preferably enzymatic saccharification which employs no chemicals such as acids. For enzymatic saccharification, a saccharifying enzyme selected from the group consisting of cellulase, xylanase, β-glucosidase and a combination thereof may be used. A combination of cellulase and xylanase at a weight ratio of 1˜2:1˜2, particularly at weight ratio of 2:1 may be used The saccharifying enzyme is used in an amount of from 1 to 20 weight parts based on 100 weight parts of the biomass. The saccharification is carried out at 40˜45° C. for 6˜24 hours, for example, for 24 hours.

In the present invention, yeast, for example, Saccharomyces cerevisiae may be used as a fermentation strain to produce bioethanol. In this context, if it is known in the art, any strain may be used as exemplified by sugar-resistant strains that perform fermentation even at high sugar concentrations, thermal resistant strains that can conduct ethanol conversion even at around 40˜45° C. which is the optimal temperature for enzymatic saccharification, and recombinant strains, such as Klebsiella oxytoca P2, Brettanomyces curstersii, Saccharomyces uvzrun, Candida brassicae, which can perform both saccharification and fermentation so as to reduce the amount of expensive enzymes used and to produce a high concentration of ethanol. The fermentation may be carried out alone at 25˜30° C. and, for example, at 30° C. for 12˜24 hours, but may be concurrent with the saccharification.

### EXAMPLE 1

Hundreds of grams of pine wood chips was prepared as biomass. The pine wood chips were immersed in 400 mL of distilled water in a beaker for 6 hours with stirring.

After the beaker (in which the biomass and water were present at a weight ratio of 1:4) was placed in a microwave oven, microwaves were applied at 700 W for 15 min. In this regard, the beaker was wrapped with a polyethylene film to reduce the evaporation of water vapor, and four holes, each 5 mm in diameter, were made in the film.

After microwaving, the biomass with a water content of 70% was withdrawn from water using a 200 mesh net.

The dewatered biomass was placed in a popping machine having the structure shown in FIG. 2 and popped at 200° C. under a pressure of 20 kg/cm³ to give a pre-treated biomass 1-1.

### EXAMPLE 2

The same procedure as in Example 1 was repeated with the exception that a wet triturating step, instead of the immersing step, was conducted, to yield pre-treated biomass 2.

In the wet triturating step, the pine wood chips were sufficiently swollen in water for a day and then, defibrated using a refiner.

Thereafter, the wet-triturated pine wood chips immersed in water in a beaker were microwaved in the same manner as in Example 1.

### EXAMPLE 3

Pre-treated biomass 3 was obtained in the same manner as in Example 2 with the exception that oak wood chips, instead of the pine wood chips, were used.

### EXAMPLE 4

Pre-treated biomass 4 was obtained in the same manner as in Example 2 with the exception that reeds, instead of the pine wood chips, were used.

### EXAMPLE 5

Pre-treated biomass 5 was obtained in the same manner as in Example 2 with the exception that flame grasses, instead of the pine wood chips, were used.

### COMPARATIVE EXAMPLE 1

The same pine wood chips as in Example 1 was ground to a size of 40˜60 mesh to yield comparative biomass 1.

### COMPARATIVE EXAMPLE 2

The same pine wood chips as in Example 1 were popped at 250° C. under a pressure of 20 kg/cm² in a popping machine to give comparative biomass 2.

### COMPARATIVE EXAMPLE 3

The same procedure as in Example 2 was repeated, with the exception that the popping step was omitted, to prepare comparative biomass 3-1 to 3-4 from pine wood chips, oak tree wood chips, reeds and flame grasses, respectively.

COMPARATIVE EXAMPLE 4

The same pine wood chips as in Example 1 were microwaved at 700 W for 15 min to give comparative biomass 4.

### EXPERIMENTAL EXAMPLE 1

The pre-treated biomass 1 obtained in Example 1 and the comparative biomass 1 obtained in

Comparative Example 1 were analyzed for monosaccharide content and the results are summarized in

Table 1, below.

TABLE 1

| (%) | Rhamnose | Arabinose | Xylose | Mannose | Galactose | Glucose | Total |
|---|---|---|---|---|---|---|---|
| C. biomass 1 | 0.5 | 1.9 | 6.1 | 11.9 | 2.7 | 54.0 | 77.1 |
| Pre-treated | 0.5 | 1.1 | 4.1 | 5.8 | 1.3 | 55.2 | 68.0 |
| biomass 1 | | | | | | | |

As seen in Table 1, the pre-treated biomass 1 according to the present invention was slightly decreased in the content of xylose, mannose and galactose, compared to the comparative biomass 1 (control), but had a glucose content similar to that of the comparative biomass 1. Thus, the complex pre-treatment of the present invention was found to slightly reduce hemicellulose content, but to have no influences on cellulose content

### EXPERIMENTAL EXAMPLE 2

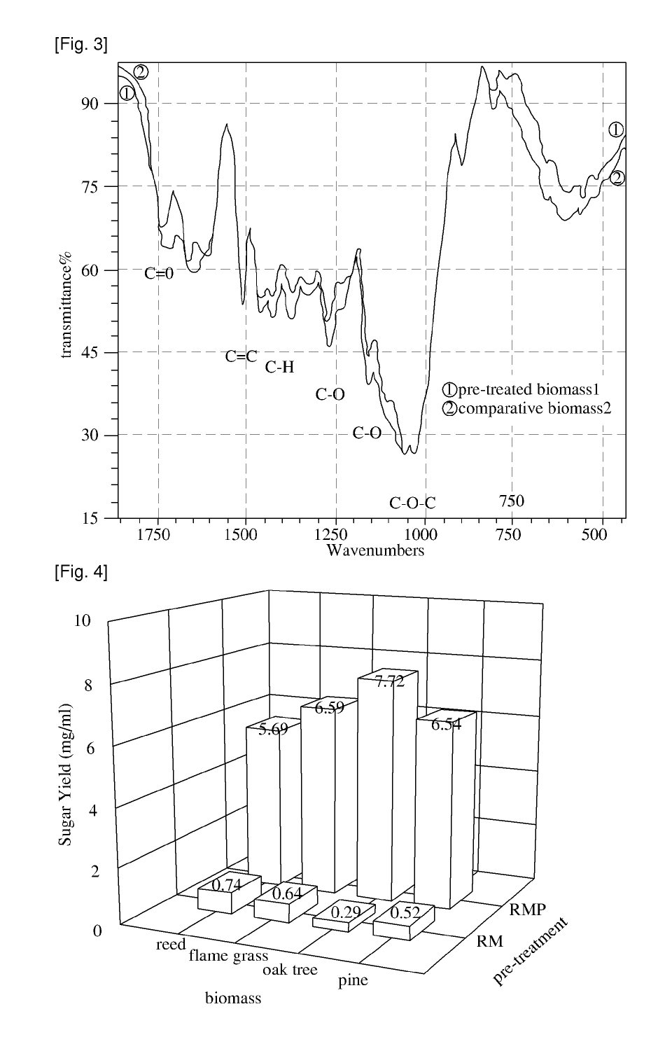

Chemical properties of the pre-treated biomass 1 obtained in Example 1 and the comparative biomass 1 obtained in Comparative Example 1 were analyzed. In this regard, the cell wall components of these were examined using FT-IR and the results are shown in FIG. 3.

Upon the pre-treatment, as seen in FIG. 3, the absorbance at 1030˜1060 cm⁻¹, which corresponds to cellulose, did not change while a reduction was detected in the absorbance at 1735 and 1157 cm⁻¹ which is featured by hemicelluloses. This FT-IR spectrum shows that the pre-treatment of the present invention causes lignocellulosic and perennial herbaceous biomass to undergo a structural change. Also, the absorbance at 1300˜1520 cm⁻¹, characterized for lignins, was also decreased. This result was coincident with the chemical analysis of Table 1.

Taken together, the experimental data obtained above demonstrate that the lignocellulosic biomass pre-treated according to the present invention is not significantly different in the content of hemicellulose and lignin from non-treated biomass, but has undergone a significant physical and structural change.

### EXAMPLE 5

To 1.50 mg of the pre-treated biomass obtained in Example 1 were added 600 U/sub.g of cellulase and 300 U/sub.g of xylase, followed by saccharification at 37° C. for 96 hours to afford saccharide 1 (wet-triturated pine wood).

### EXAMPLE 6

The same procedure as in Example 5 was repeated, with the exception that pre-treated biomass 2 was used, to afford saccharide 2 (pine wood).

### EXAMPLE 7

The same procedure as in Example 5 was repeated, with the exception that pre-treated biomass 3 was used, to afford saccharide 3 (oak wood).

### EXAMPLE 8

The same procedure as in Example 5 was repeated, with the exception that pre-treated biomass 4 was used, to afford saccharide 4 (reed).

### EXAMPLE 9

The same procedure as in Example 5 was repeated, with the exception that pre-treated biomass 5 was used, to afford saccharide 5 (flame grass).

### COMPARATIVE EXAMPLE 5

To 1.50 mg of the biomass obtained in Comparative Example 1 were added 600 U/sub.g of cellulase and 300 U/sub.g of xylase, followed by saccharification at 37° C. for 96 hours to afford comparative saccharide 1.

### COMPARATIVE EXAMPLE 6

The same procedure as in Comparative Example 5 was repeated, with the exception that comparative biomass 2 obtained in Comparative Example 2 was used, to afford comparative saccharide 2 (pine wood).

### COMPARATIVE EXAMPLE 7-1

The same procedure as in Comparative Example 5 was repeated, with the exception that comparative biomass 3-1 obtained in Comparative Example 3 was used, to afford comparative saccharide 3-1 (pine wood).

### COMPARATIVE EXAMPLE 7-2

The same procedure as in Comparative Example 5 was repeated, with the exception that comparative biomass 3-2 was used, to afford comparative saccharide 4 (acorn wood).

### COMPARATIVE EXAMPLE 7-3

The same procedure as in Comparative Example 5 was repeated, with the exception that comparative biomass 3-3 was used, to afford saccharide 3-3 (reed).

### COMPARATIVE EXAMPLE 7-4

The same procedure as in Comparative Example 5 was repeated, with the exception that comparative biomass 3-4 was used, to afford saccharide 3-4 (flame grass).

### COMPARATIVE EXAMPLE 8

The same procedure as in Comparative Example 5 was repeated, with the exception that comparative biomass 4 was used, to afford saccharide 4.

### EXPERIMENTAL EXAMPLE 3

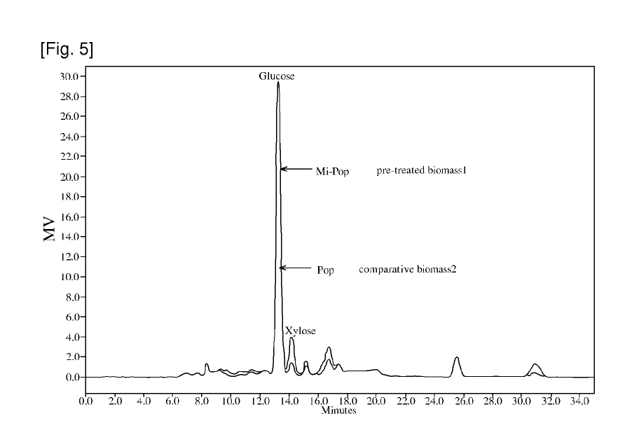

To analyze the effect of the pre-treatment of the present invention on the saccharification yield of enzymes, the contents of the reducing sugar for each of the saccharides 1 to 5 obtained in Examples 5 to 9 were compared to those in the comparative saccharides 1 to 4 obtained in Comparative Example 5 to 8, using a DNS method. HPLC was performed to analyzing the contents of monosaccharides. The analysis results of saccharides 3 to 5 are graphically presented, together with those of comparative saccharides 3-1 and 3-2, in FIG. 4.

After enzymatic saccharification, as shown in FIG. 4, sugar contents were measured to be 0.52 mg/mL in comparative saccharide 3-1 (pine wood), 0.29 mg/mL in comparative saccharide 3-2 (oak wood), 0.74 mg/mL in comparative saccharide 3-3 (reed), and 0.64 mg/mL in comparative saccharide 3-4 (flame grass). On the other hand, sugar contents were increased when the pre-treatment including wet-triturating, microwaving and popping was performed: 6.54 mg/mL in saccharide 2 (pine wood); 7.72 mg/mL in saccharide 3 (oak); 5.69 mg/mL in saccharide 4 (reed); and 6.59 mg/mL in saccharide 5 (flame grass).

Therefore, the complex pre-treatment including wet-triturating, microwaving and popping in accordance with the present invention increased the sugar content by 670 fold for reeds, 930 fold for flame grasses, 2560 fold for oak wood and 1157 fold for pine woods, compared to that in the controls (comparative saccharides 3-1 to 3-4). Saccharide 1 (wet-triturated pine wood), obtained in Example 5, was found to have a sugar content of 6.455 mg/mL, demonstrating the advantage of the pre-treatment of the present invention.

Although not shown, sugar contents were measured to be 0.545 mg/mL in comparative saccharide 1, 2.045 mg/mL in comparative saccharide 2, and 0.555 mg/mL in comparative saccharide 4. As is apparent from the data, both the microwaved group (comparative saccharide 4) and the non-treated group (comparative saccharide 1) show a hydrolysis rate of approximately 0.5 mg/mL, indicating that microwaving alone cannot improve the saccharification yield and popping (comparative saccharide 2), although effective to some degree, cannot bring about a significant effective compared to the complex pre-treatment of the present invention.

### EXPERIMENTAL EXAMPLE 4

The glucose contents of Saccharide 1, obtained in Example 5, and comparative saccharide 1, obtained in Comparative Example 6 were analyzed and the results are shown in FIG. 5.

As seen in the spectrum of FIG. 5, a much higher glucose peak was detected in saccharide 1 than comparative saccharide 1. Coincident with the result of Experimental Example 3, these data indicate that the pre-treatment of the present invention including wet-triturating, microwaving and popping significantly improves the saccharification yield of lignocellulosic and perennial herbaceous biomass.

Taken together, the results of the experiment demonstrate that lignocellulosic biomass (pine woods, oak wood) and perennial herbaceous biomass, especially reeds and flame grasses, all of which are difficult to saccharify, can be converted into those suitable for use in bioengineering processes by the thermophysical treatment of the present invention alone, without chemical treatment

### EXAMPLE 10

Production of Bioethanol

1. Hydrolytic Pre-treatment Process

The same procedure as in Example 1 was repeated to give pre-treated biomass 1.

2. Saccharification Process

The same procedure as in Example 5 was repeated to give a saccharide 1, that is, glucose.

3. Fermentation Process

The glucose obtained above was concentrated into 10%, and the concentrate was added in an amount of 15 g/L to a solution of Saccharomyces cerevisiae, a fermentation strain for the production of bioethenol, and incubated at 30° C. for 24 hours to produce bioethanol.

The saccharification process and the fermentation process may be performed simultaneously.

Although the exemplary embodiments of the present invention have been disclosed for illustrative purposes, those skilled in the art will appreciate that various modifications, additions and substitutions are possible, without departing from the scope and spirit of the invention as disclosed in the accompanying claims.

1. A method for hydrolytic pre-treatment of lignocellulosic and perennial herbaceous biomass, comprising:
preparing lignocellulosic biomass or perennial herbaceous biomass in form of chips, said lignocellulosic biomass comprising pine wood and oak wood, said perennial herbaceous biomass comprising reeds and flame grasses;
immersing the biomass in water or triturating the biomass in a wetting manner;
microwaving the hydrated biomass or the wet-triturated biomass for 10 to 40 min at 500 W to 800 W;
dewatering the microwaved biomass; and
popping the dewatered biomass under a pressure of 5˜30 kgf/cm² in a popping machine.

2. (canceled)

3. The method of claim 1, wherein the immersing step comprises immersing the biomass in water for 3 hours or longer and stirring 100 wt parts of the biomass in 300˜700 wt parts of water.

4. The method of claim 1, wherein the triturating step comprises swelling the biomass for 12 hours in water and triturating the swollen biomass to defibration.

5. The method of claim 1, wherein the popping machine comprises:
a popping tank for accommodating the wet-triturated biomass, designed to maintain high temperatures and high pressures therein when heated;
a direct fired burner for applying heat directly to the popping tank;
a storage tank having a space for storing the popped biomass, into which a part of the popping tank is detachably attached;
a motor for rotating the popping tank directly heated by the direct fired burner, whereby a temperature and a steam distribution can be maintained constant throughout the popping tank; and
a controller for controlling the popping tank in terms of either or both of pressure and temperature.

6. The method of claim 5, wherein the popping tank is equipped with a temperature sensor and/or a pressure sensor therein.

7. A method for production of a saccharide from lignocellulosic and perennial herbaceous biomass, comprising saccharifying the lignocellulosic and perennial herbaceous biomass pre-treated by the method of claim 1.

8. The method of claim 7, wherein the saccharifying step is carried out by incubating 100 weight parts of the biomass in the presence of 1 to 20 weight parts of a saccharifying enzyme.

9. The method of claim 8, wherein the saccharifying enzyme is selected from the group consisting of cellulase, xylanase, β-glucosidase and a combination thereof.

10. A method for production of bioethanol from lignocellulosic and perennial herbaceous biomass, comprising:
pre-treating the lignocellulosic and perennial herbaceous biomass using the method of claim 1;
saccharifying the pre-treated biomass to give a saccharide; and
fermenting the saccharide.

11. The method of claim 10, wherein the saccharifying step and the fermenting step are conducted simultaneously.

12. The method of claim 11, wherein the saccharifying step and the fermenting step are conducted after the pre-treated biomass is treated with a recombinant strain selected from the group consisting of Klebsiella oxytoca P2, Brettanomyces curstersii, Saccharomyces uvzrun, and Candida brassicae.
